(19) United States
(12) Patent Application Publication
(10) Pub. No.: US 2007/0003987 A1
(43) Pub. Date: Jan. 4, 2007

(54) Screening method for substance binding to merozoite surface protein-1/42

(75) Inventors: Anton Richard Dluzewski, London (GB); Anthony Arthur Holder, London (GB); Berry Martin Birdsall, London (GB); Jeff James Babon, London (GB); Stephen Richard Martin, London (GB); William Dale Morgan, London (GB); James Feeney, London (GB); Michael John Blackman, London (GB); Suzanne Lesley Fleck, London (GB); Barbara Saxty, London (GB)

Correspondence Address: MORGAN LEWIS & BOCKIUS LLP, 1111 PENNSYLVANIA AVENUE NW, WASHINGTON, DC, 20004, US

(73) Assignees: MEDICAL RESEARCH COUNCIL, London (GB); KING'S COLLEGE LONDON, London (GB)

(21) Appl. No.: 10/561,196

(22) PCT Filed: Jun. 18, 2004
(86) PCT No.: PCT/GB04/02604

§ 371 Date: Jun. 27, 2006

(30) Foreign Application Priority Data
Jun. 18, 2003 (GB) 0314114.0

(51) Int. Cl. G01N 33/569; A61K 31/185

(52) U.S. Cl. 435/7.22; 514/553

(57) ABSTRACT

Disclosed is a method of screening a test substance for possession of binding activity for possession of binding activity for MSP₄₂ or a fragment thereof, the method comprising the steps of: combining or contacting, in any order, (i) a molecule comprising MSP1₄₂ or a fragment thereof, (ii) the test substance, and (iii) a comparison substance known to have binding activity for MSP1₄₂ or a fragment thereof; and determining the presence and/or amount, if any, of comparison substance and/or test substance bound of the MSP1₄₂ or fragment thereof.

### FIELD OF THE INVENTION

The present invention relates to a method of screening a test substance for possession of binding activity for MSP1₄₂. In preferred embodiments the invention provides a method of screening a test substance to identify those which have the ability to inhibit or otherwise interfere with the natural protease-mediated processing of MSP1₄₂ into MSP1₃₃ and MSP1₁₉ fragments.

### BACKGROUND OF THE INVENTION

The protozoon Plasmodium falciparum develops and replicates within erythrocytes, releasing merozoites that invade new red blood cells. This stage of the parasite's life cycle is responsible for the disease malaria, and inhibition of merozoite invasion reduces parasitaemia, with beneficial outcome for the host.

Several proteins have been identified on the surface of the merozoite (Holder, 1994 Parasitology 108, Suppl. S5-18). For example, P. falciparum MSP1 (merozoite surface protein 1) is synthesised as a ^{˜}200 kDa precursor and is present on the surface of the late stage parasite within the erythrocyte. At or immediately prior to merozoite release, MSP1 is cleaved (primary processing) into four fragments that form part of a protein complex on the surface of the free merozoite. One of these fragments, the C-terminal 42 kDa-polypeptide (MSP1₄₂), has a GPI-anchor holding the complex to the parasite surface. At erythrocyte invasion the protein complex is released from the merozoite surface following secondary processing involving a single proteolytic cleavage within MSP1₄₂. The 33 kDa N-terminal part of MSP1₄₂ is shed with the complex whereas MSP1₁₉, the C-terminal part of MSP1₄₂, remains on the surface of the invading merozoite. MSP1₁₉ contains two epidermal growth factor (EGF) domains (Morgan et al, 1999 J. Mol. Biol. 289, 113-122). Certain MSP1₁₉-specific monoclonal antibodies (mAbs) inhibit both secondary processing and erythrocyte invasion (Blackman et al, 1994 J. Exp. Med. 180, 389-393) suggesting that inhibitors of the protease responsible for MSP1 secondary processing inhibit invasion. Prevention of secondary processing of MSP1 may thus be a good chemotherapeutic target.

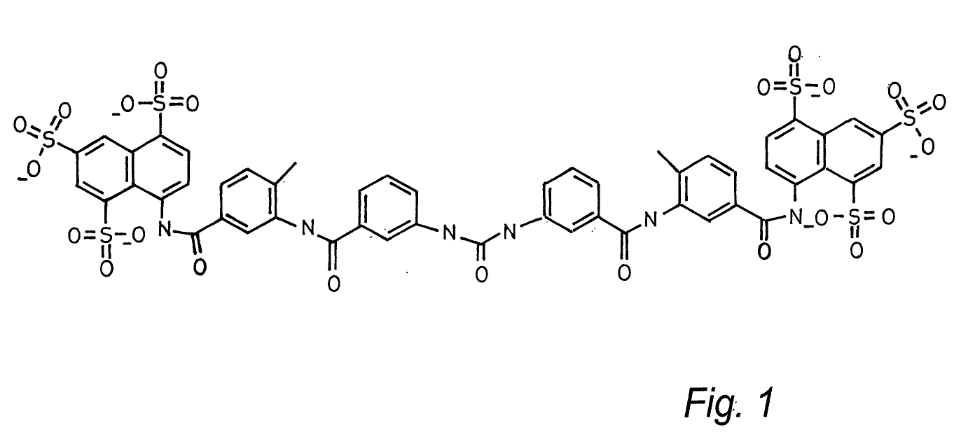

Suramin is a polysulfonated naphthyl urea that has been used for many years as a trypanocide in the treatment of sleeping sickness and more recently as a treatment for filariasis (Hawking 1978 Adv. Pharmacol. Chemother. 15, 289-322). Suramin is a symmetrical molecule and highly negatively charged. The structure of the compound is illustrated schematically in FIG. 1. It has multiple biological effects in vivo, reviewed by, for example, Scher & Kelly (PPO Updates 1993 7, 1-16).

In addition to the clinical usage noted above, suramin has been the subject of several clinical trials because of reported antiviral and antitumour effects. However, suramin is not widely used therapeutically because it has a narrow therapeutic window, being highly toxic and associated with unpleasant undesirable side effects (Voogol et al, 1993 Pharmacol. Rev. 45, 177-203).

In addition to suramin per se, numerous analogues of suramin have been synthesised, some of which have also been the subject of clinical trials, in an attempt to find substances which have the efficacy of suramin but with less toxicity. Many suramin analogues have been disclosed and described by Firsching-Hauk et al, (2000 Anti-Cancer Drugs 11, 69-77) and by Dhar et al, (2000 European Journal of Cancer 36, 803-809).

There is one report (Dluzewski et al, “Inhibition of Malaria Invasion by Extracellular ATP Analogues”; in Molecular Approaches to Malaria, 2^{nd}-5^{th} Feb. 2000, Erskine House, Lorne, Victoria, Australia, Ed. M. Macreadie, ISBN 0 646 38983 1) that suramin inhibits merozoite invasion of erythrocytes, although the mechanism was unknown and not disclosed.

### SUMMARY OF THE INVENTION

In a first aspect the invention provides a method of screening a test substance for possession of binding activity for MSP1₄₂ or a fragment thereof, the method comprising the steps of: combining or contacting, in any order,
(i) a molecule comprising MSP1₄₂ or a fragment thereof,
(ii) the test substance, and
(iii) a comparison substance known to have binding activity for MSP1₄₂ or a fragment thereof;
   and determining the presence and/or amount, if any, of comparison substance and/or test substance bound to the MSP1₄₂ or fragment thereof. The method may provide results in either a qualitative or a quantitative manner. Not only can the method of the invention be used to identify compounds which are able to bind to MSP1₄₂ or a fragment thereof, but may alternatively or additionally be used to characterise such binding (e.g. measurement of absolute or relative binding affinity; or identification of the binding site on MSP1₄₂—by using different fragments or variants of MSP1₄₂).

The MSP1₄₂ used in the method of the invention may be, for example, derived from Plasmodium parasites and purified or, more preferably, may be recombinant MSP1₄₂ expressed in, for example, a bacterial, yeast or eukaryotic cell culture in the absence of Plasmodium parasites. The MSP1₄₂ may, in theory, be present as part of a larger molecule (e.g. the MSP1 200 kDa precursor molecule) but this is not preferred as the possibility of binding of the test substance to the MSP molecule outside the MSP1₄₂ portion would need to be prevented or excluded from consideration in some way. Accordingly, it is preferred that the molecule used in the method of the invention is substantially limited to MSP1₄₂ or a fragment thereof, and any other portions of the MSP1 molecule are restricted to components which are known not to bind to the test substance. Nevertheless, additional components may be desirable to include in, or attach to, the MSP1₄₂ molecule e.g. to facilitate immobilisation to a solid surface such as the well of a microtitre plate or other substrate. If deemed appropriate, any additional component included in or attached to the MSP1₄₂ molecule could be separately tested for binding to the test substance, so as to exclude such a possibility or allow for such binding in consideration of the assay results.

The method of the present invention also allows for use of a fragment of MSP1₄₂. The fragment will preferably comprise at least 50 amino acids, more preferably at least 100 amino acids, and most preferably at least 150 amino acids. Desirably the fragment comprises one of the naturally-occurring fragments derived by protease degradation: MSP1₁₉ and MSP1₃₃. The MSP1₁₉ fragment is derived from the C terminal portion of MSP1₄₂ and the MSP1₃₃ fragment is derived from the N terminal portion of MSP1₄₂.

The MSP1₄₂ molecule or fragment thereof may be a naturally-occurring molecule (that is, comprise an amino acid sequence identical to that encoded by a Plasmodium organism) or may be slightly different (e.g. comprise a few, say, less than 20, amino acid residue substitutions, preferably less than 10 substitutions) relative to a naturally-occurring molecule. Thus, for example, the MSP1₄₂ molecule or fragment thereof may be a mutant of a naturally-occurring wild type sequence. The mutant may be a spontaneous mutant or a laboratory-induced mutant. The MSP1₄₂ molecule or fragment thereof may also comprise substitutions and/or additional residues e.g. to facilitate expression or purification.

If desired, the test substance can be assayed or screened against a number of different MSP1₄₂ molecules (or fragments thereof) so as to provide extra information e.g. about the binding site and/or other properties of the interaction with MSP1₄₂ or the fragment thereof.

The MSP1₄₂ molecule or fragment thereof may be derived from, correspond to or be similar to that obtainable from any Plasmodium species including, for example, P. cynomolgi, P. knowlesi, P. berghei, P. chabaudi, or P. yoelii. It is preferred, however, that the MSP1₄₂ molecule or fragment thereof may be derived from, correspond to or be similar to that obtainable from one of the Plasmodium species known to be capable of infecting humans i.e. P. falciparum, P. vivax, P. malariae and P. ovale. Of these, falciparum and vivax are preferred.

The test substance may be any substance of potential interest. Preferably the test substance is a compound or mixture of potential therapeutic interest and is therefore preferably of low toxicity for mammals. In particular the test substance may form part of a library of substances, e.g. a library produced by combinatorial chemistry, or a phage display library.

The general principle of the method of the invention is that of a competition assay—if the test substance is able to bind to MSP1₄₂ or a fragment thereof, and more especially if it binds at or close to the same site as that bound by the comparison substance, then the presence of the test substance will compete with, interfere or inhibit the binding of the comparison substance to the MSP1₄₂ molecule or fragment thereof.

The binding affinity of the test and comparison substances may be quite different, and it may be desirable therefore to perform the method using a variety of ratios of test and comparison substance concentrations. The method may be performed by contacting the test and comparison substances substantially simultaneously with the MSP1₄₂ molecule or fragment thereof. Alternatively, one of the substances may be pre-incubated with the MSP1₄₂ molecule or fragment thereof, and the other substance introduced subsequently to see if any displacement of pre-bound test or comparison substance, as appropriate, takes place.

The step of determining the amount of bound test and/or comparison substance may be achieved using any of the numerous suitable assay techniques known to those skilled in the art, such as radioassay, fluorescence assay, ELISA, isothermal titration calorimetry (ITC), surface plasmon resonance (SPR) and the like. The assay is preferably one which is amenable to automation and/or high throughput screening. Desirably the assay is performed on a disposable solid support such as a microtitre plate or similar.

The comparison substance may be any substance which is known to bind to MSP1₄₂. The comparison substance may be, for example, an antibody or antigen-binding variant thereof (such as an Fab, Fv, scFv etc), a peptide or synthetic chemical compound. The comparison substance may conveniently be labelled with a readily detectable marker, which serves to facilitate detection of the labelled comparison substance and hence determination of the amount bound (or unbound) to the MSP1₄₂ molecule or fragment thereof, although this is not essential. The label may comprise, for instance, a radio label, an enzyme label, an antibody label, a fluorescent label, a particulate (e.g. latex) label or the like.

The present inventors have identified substances which, upon binding to MSP1₄₂, inhibit the normal protease-mediated processing of the molecule into MSP1₃₃ and MSP1₁₉ fragments, which processing is an essential part of the pathway by which Plasmodium merozoites invade erythrocytes. Thus, by using such a substance as the comparison substance, the present invention provides a method of identifying test substances which will bind to the same or similar portion of the MSP1₄₂ molecule and hence should similarly inhibit processing of MSP1₄₂ into MSP1₃₃ and MSP1₁₉ and/or inhibit merozoite invasion of erythrocytes. Accordingly in preferred embodiments the comparison substance is a substance which, upon binding to MSP1₄₂, may inhibit the normal protease-mediated processing of the molecule into MSP1₃₃ and MSP1₁₉ fragments. In such an embodiment the method of the invention provides, in effect, a method of screening a test substance for the ability to interfere with or inhibit secondary processing of MSP1₄₂ and/or inhibit merozoite invasion of erythrocytes. In this way the method of the invention can be used to identify/screen drug-like compounds with potential application as anti-malarials and which are less toxic than suramin.

One such group of substances is constituted by suramin and various analogues thereof. The structure of suramin is shown in FIG. 1. The inventors have found that suramin and analogues thereof bind to MSP1₄₂ or fragments thereof derived from, inter alia, P. falciparum, P. vivax and P. yoelii. In preferred embodiments of the invention, the comparison substance comprises suramin or, more preferably, an analogue of suramin.

The symmetrical nature of the suramin molecule means that some analogues comprise structural variants based on just ½ of the suramin molecule (comprising 4 aromatic rings and half of the anionic substituents). Such analogues may comprise just one or two sulphonyl groups (instead of the three sulphonyl groups present on ½ the suramin molecule). The sulphonyl groups may be present at different positions to those that they occupy in suramin. Alternatively, anionic, cationic, or uncharged groups may be present instead of, or in addition to, the sulphonyl groups. The same comments apply to variants which are structural analogues of the whole suramin molecule.

Accordingly, the term “suramin analogue”, for the purposes of the present invention, encompasses all molecules with at least two aromatic ring(s), the rings comprising, between them, at least one, preferably at least two, and more preferably at least three, uncharged or charged (preferably negatively charged) substituent groups. The suramin analogue will desirably be able to bind to MSP1₄₂ or a fragment thereof with Kd (a measure of affinity) of 5 μM or lower, as determined by fluorimetry assay or isothermal titration calorimetry, according to the methods detailed in Example 3 below.

The substituent group(s) attached to the aromatic ring are preferably negatively charged under the conditions in which the assay method of the invention is performed. The substituent group may be a sulphonyl group (which is preferred), as in the suramin molecule, or may be any other suitable group of generally similar size and charge.

Conveniently, a suramin analogue will be symmetrical, and will typically comprise two conjoined aromatic rings at each end of the molecule (i.e. four rings in total) joined by a linker group comprising urea or (more preferably) a urea derivative.

Preferably the two or more aromatic rings will be covalently bonded to urea or a urea derivative comprising the moiety —NHCONH—, such as m-aminobenzoyl 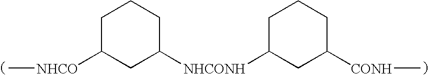
or m¹-aminobenzoyl-m-aminobenzoyl.

In preferred embodiments the suramin analogue comprises two or more aromatic rings, with negatively charged substituents, attached (directly or indirectly) to one end of a molecule of urea or a urea derivative, and two or more aromatic rings, with negatively charged substitutents, attached (directly or indirectly) to the other end of the molecule of urea or urea derivative. Compounds of the type shown in groups B and C of Table 1 represent examples of preferred suramin analogues.

Suramin and many of its analogues possess intrinsic fluorescence. Accordingly, it is possible to determine binding of suramin or a suramin analogue to e.g. MSP1₄₂ by measuring the amount of intrinsic suramin-mediated or suramin analogue-mediated fluorescence in an assay system. Alternatively, the suramin or suramin analogue may be labelled with a conventional label moiety and the presence and/or amount of binding determined by detection and/or measurement of the conventional label.

More especially, the inventors have discovered that some analogues of suramin exhibit greatly increased fluorescence when bound to MSP1 (much greater fluorescence than that exhibited by suramin itself). These analogues include those identified in Table 1 as C2 and C4. The inventors have further shown that this fluorescence is significantly reduced in the presence of a competitor substance (e.g. suramin) which competes for binding to MSP1. Thus a preferred method of the invention comprises use of a comparison substance which exhibits one level of fluorescence when bound to a molecule comprising MSP1₄₂ or a fragment thereof, and a detectably different level of fluorescence when not so bound (e.g. when displaced from MSP1₄₂ or prevented from binding thereto by a competitor molecule. Advantageously, such alteration in the level of fluorescence of the comparison substance is substantially specific as regards binding to a molecule comprising MSP1₄₂ relative to binding to other substances. Preferably there is at least a two fold, more preferably at least a threefold, difference in the relative fluorescence levels. Suitable comparison substances for use in such a preferred embodiment include, but are not limited to, compounds C2 and C4 in Table 1. Whilst some of the suramin analogues investigated by the inventors undergo a marked increase in fluorescence upon binding to MSP1, in theory, molecules which exhibit a marked decrease in fluorescence upon binding to MSP1 might also be useful in the present invention.

The present inventors have found that suramin binds relatively tightly to MSP1₄₂ and MSP1₃₃, but relatively weakly to MSP1₁₉. They have further identified particular residues in MSP1₁₉ which may be involved in suramin binding.

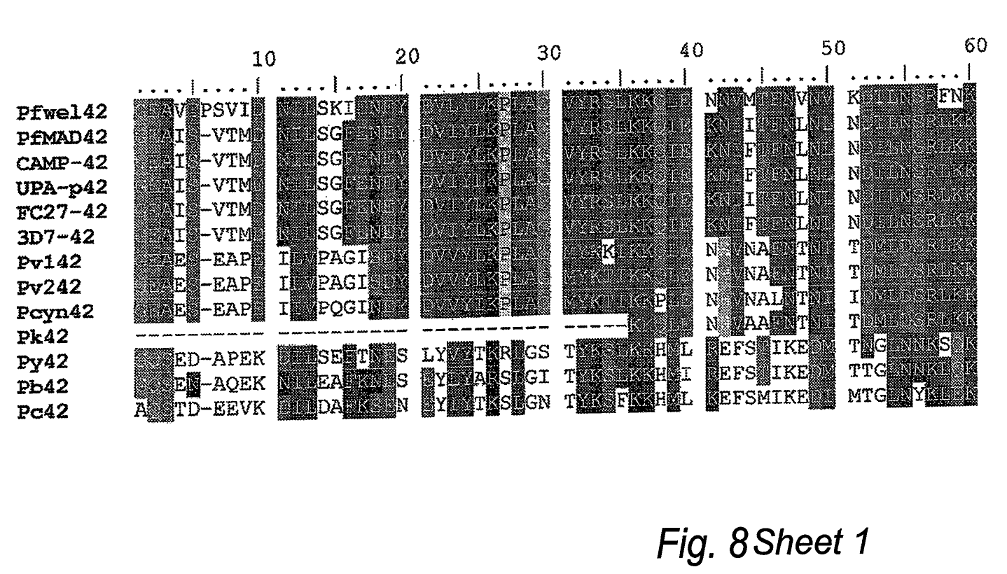

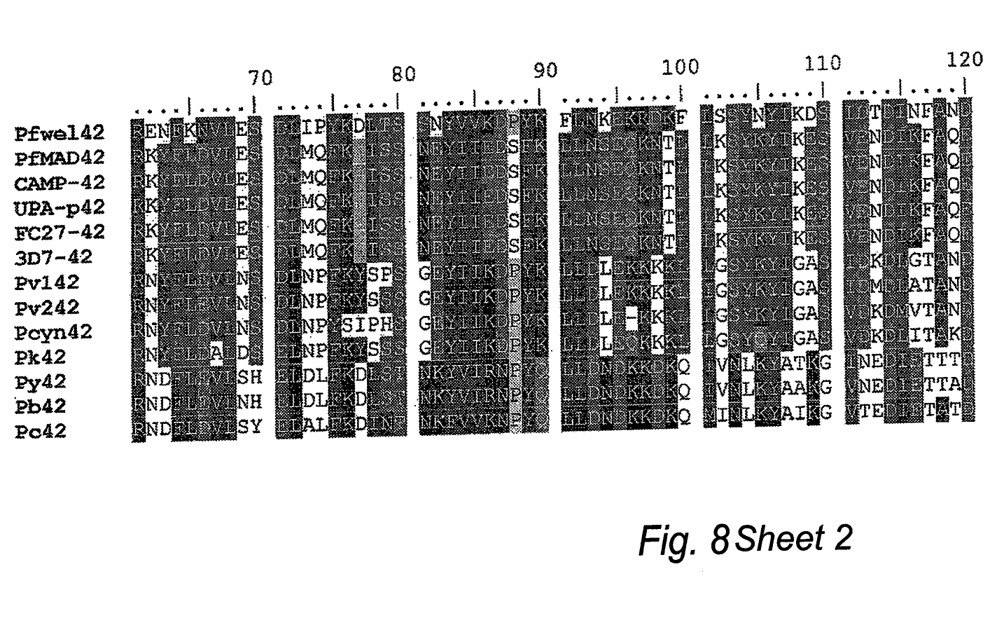

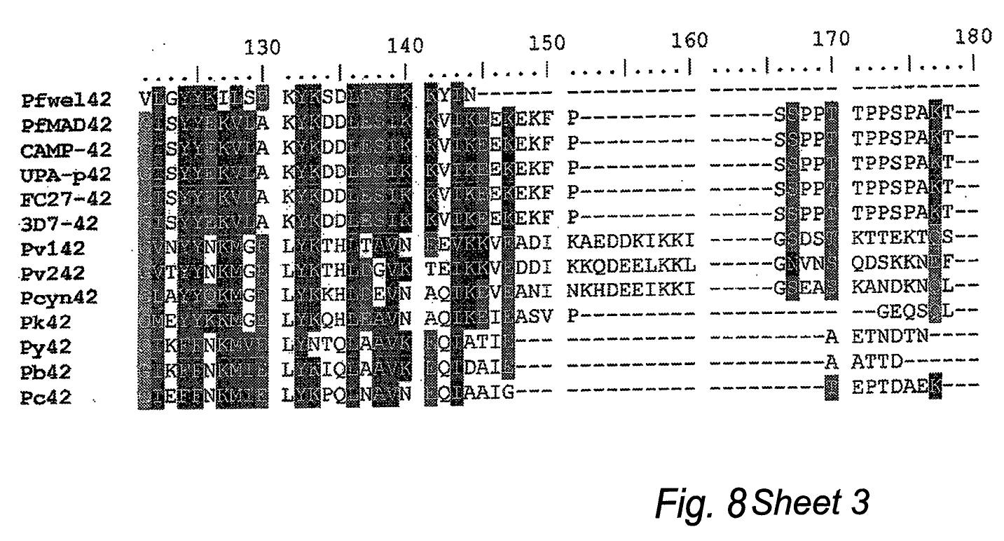

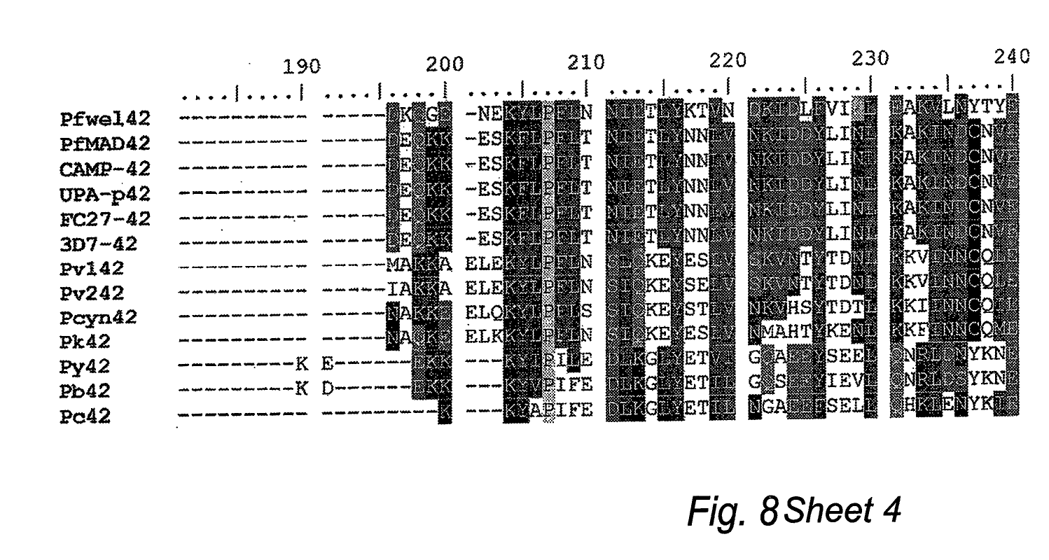

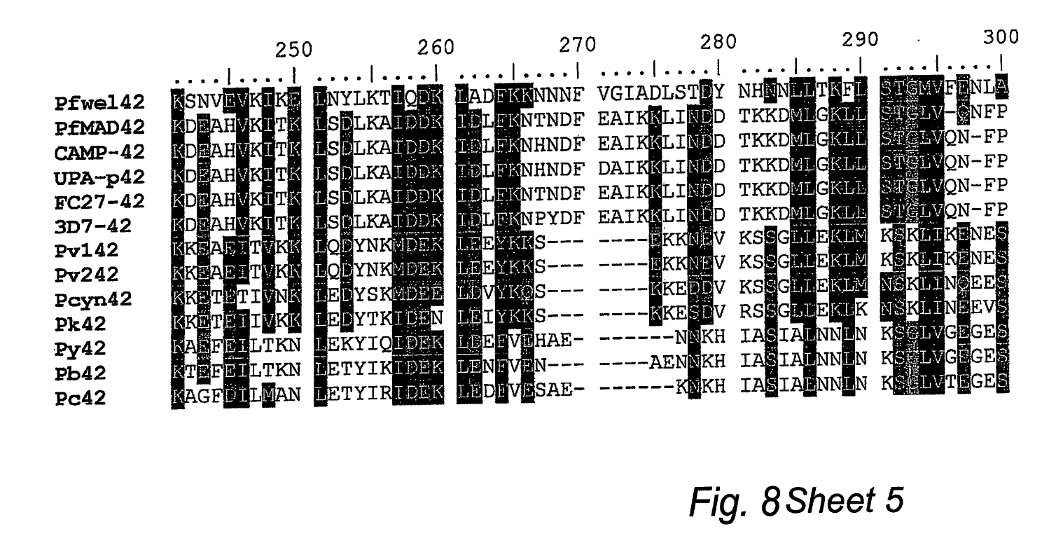

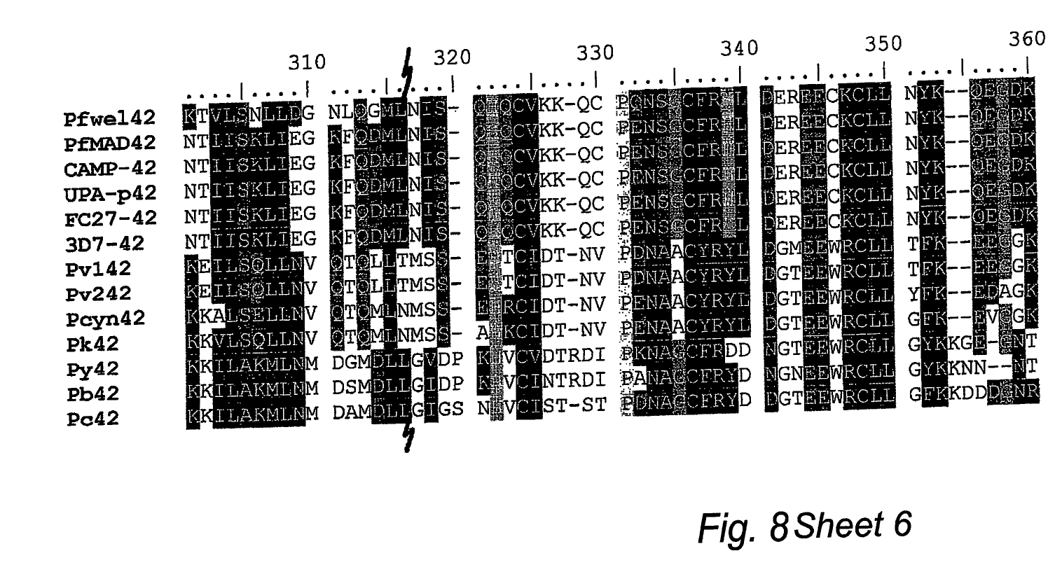

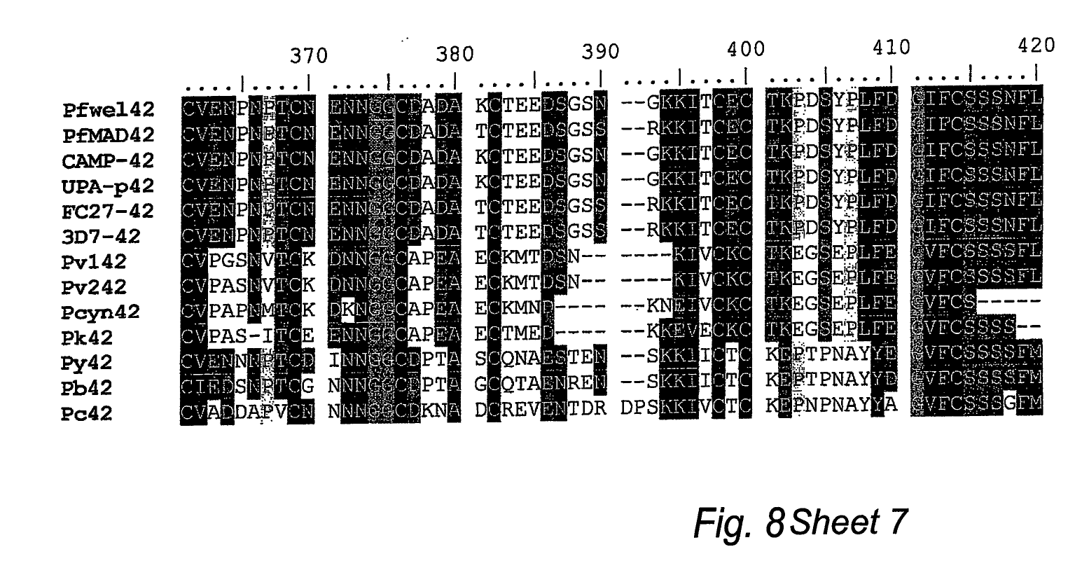

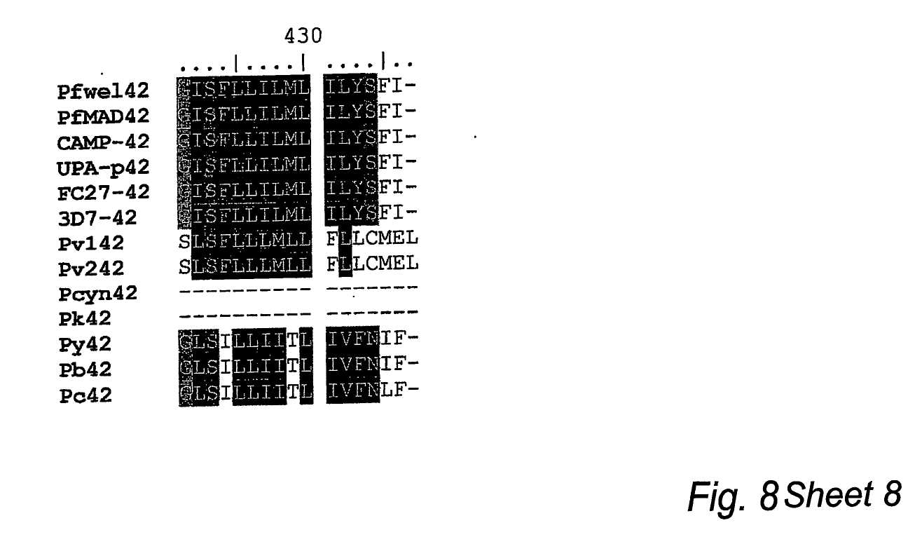

FIG. 8 shows alignment of the amino acid sequences of MSP1₄₂ protein from various Plasmodium sp.

In FIG. 8 Pfwel, PfMAD, CAMP, UPA, FC27 and 3D7 are all P. falciparum strains; PV1 and PV2 are P. vivax strains; Pcyn is P. cynomolgi; PK is P. knowlesi; Py is P. yoelii; Pb is P. berghei and Pc is P. chabaudi. The table below sets out the accession numbers in the GenBank/EMBL/DDJB databases of the various amino acid sequences.

| | | Sequence |
|---|---|---|
| Parasite species | Parasite line | accession number |
| Plasmodium falciparum | Wellcome | X02919 |
| Plasmodium falciparum | MAD 20 | X05624 |
| Plasmodium falciparum | CAMP | X03831 |
| Plasmodium falciparum | Uganda-Palo Alto (UPA) | M37213 |
| Plasmodium falciparum | FC27 | M19143 |
| Plasmodium falciparum | 3D7 | Z35327 |
| Plasmodium vivax | Belem | AF435594 |
| Plasmodium vivax | Sal-1 | M75674 |
| Plasmodium cynomolgi | Ceylonesis | U25743 |
| Plasmodium knowlesi | Nuri | X91855 |
| Plasmodium yoelii | YM | J04668 |
| Plasmodium berghei | K173 | U43521 |
| Plasmodium chabaudi | IPP | M34947 |

The cleavage site between MSP1₃₃ and MSP1₁₉ is, in the alignment, between residues 316 and 317 and is indicated by a jagged arrow, such that residue 317 of MSP1₄₂ corresponds to residue 1 of MSP₁₉. The shading represents various blocks of conserved sequence.

The inventors have found, in particular, that the following residues of MSP₁₉ of P. falciparum are implicated in interaction with suramin: I2, H5, F19, H21, L22 and R25. Significantly, H5 is conserved across all species, F19 is semi-conserved (also appearing as Y19) and L22 is widely conserved. Accordingly, it is likely that substances (either test substances or comparison substances) which interact with at least one, two, three, four, five or six (in increasing order of preference) of the MSP1₁₉ residues identified above will similarly inhibit MSP1₄₂ processing and/or merozoite invasion of erythrocytes.

The assay may involve simple detection and/or measurement of comparison and/or test substance bound to the MSP1₄₂ molecule or fragment thereof (in a “direct” binding assay), but such binding may be detected indirectly e.g. by determining any inhibitory effect on merozoite invasion of erythrocytes and/or inhibition of MSP1₄₂ processing. Generally, a direct binding assay will be more amenable to automation and/or high throughput screening techniques.

Methods similar to those used by the inventors and described in the present specification may, with the benefit of the present disclosure, be employed without undue effort by those skilled in the art to identify similar residues in the MSP1₃₃ fragment of MSP1₄₂ which are involved in binding to suramin or suramin analogues and, based on knowledge of the 3 dimensional structure of suramin and MSP1₄₂, this information could be used to computer model compounds which will be predicted to bind to MSP1₄₂ and may therefore inhibit MSP1₄₂ processing.

Given the highly hydrophobic nature of the suramin molecule, the inventors propose that the residues of MSP1₃₃ involved in binding to suramin are likely to be hydrophobic, so as to form a hydrophobic pocket or cleft into which the suramin molecule can become inserted. Since the inventors have found that MSP1₄₂ from both P. falciparum and P. vivax can bind suramin, it also seems reasonable to suppose that the residues will be at least semi-conserved between these species.

In a second aspect the invention provides for a method of preventing and/or treating malarial disease by administering an effective amount of suramin or, more preferably, a suramin analogue, to a mammalian (preferably human) subject in need of such treatment. More specifically the invention provides for a method of inhibiting merozoite invasion of erythrocytes in a mammalian host.

In a third aspect the invention provides for use of suramin or, more preferably, a suramin analogue, in the preparation of a medicament to prevent and/or treat malarial disease in a mammalian subject.

Preferred suramin analogues are those compounds which exhibit a degree of activity similar to that of suramin in terms of inhibiting MSP1₄₂ processing and/or inhibiting merozoite invasion of erythrocytes, but with reduced toxicity for the mammalian subject.

For the avoidance of doubt, it is hereby expressly stated that features described herein as “preferred”, “advantageous”, “convenient” or “desirable” and the like may be adopted in the invention in isolation or in combination with any one or more other features so described, unless the context dictates otherwise.

The invention will now be described further by way of illustrative example and with reference to the accompanying drawings, in which:

FIG. 1 is a schematic representation of the structure of suramin;

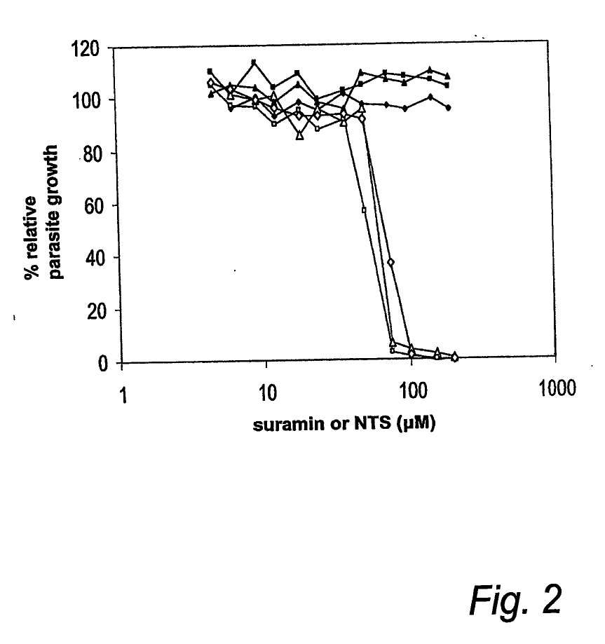

FIG. 2 is a graph showing % relative parasite growth in the presence of suramin or NTS at various concentrations;

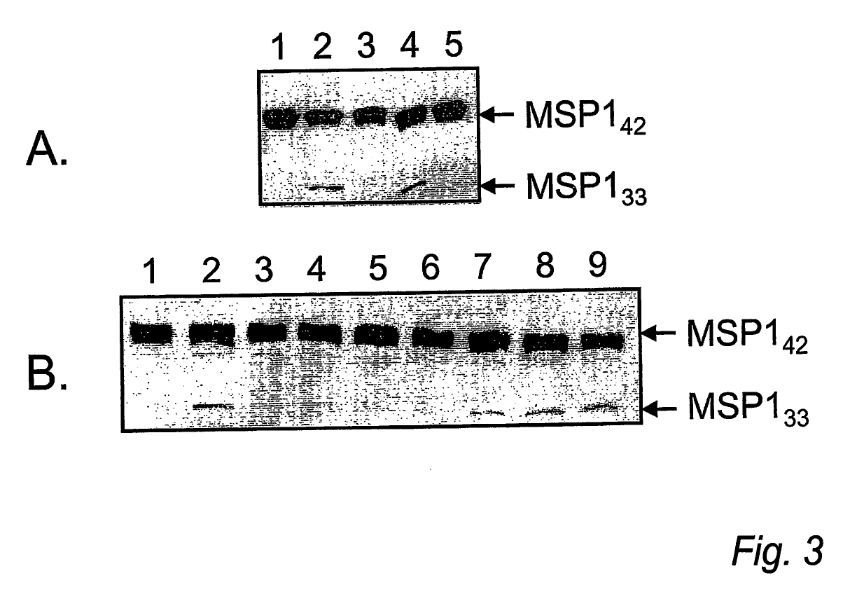

FIGS. 3A and 3B are pictures of Western blots of MSP1 proteins subjected to various treatments;

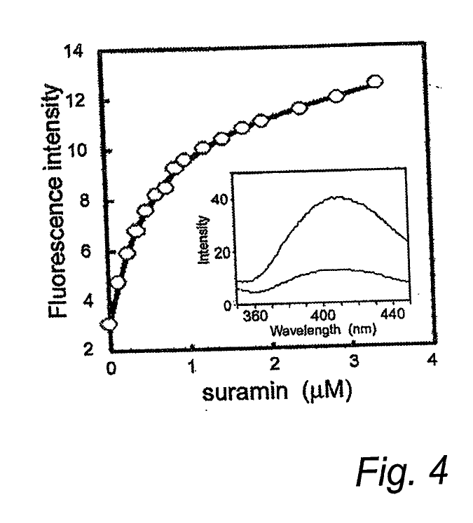

FIG. 4 is a graph showing fluorescence intensity (arbitary units) for suramin at various concentrations interacting with MSP 1;

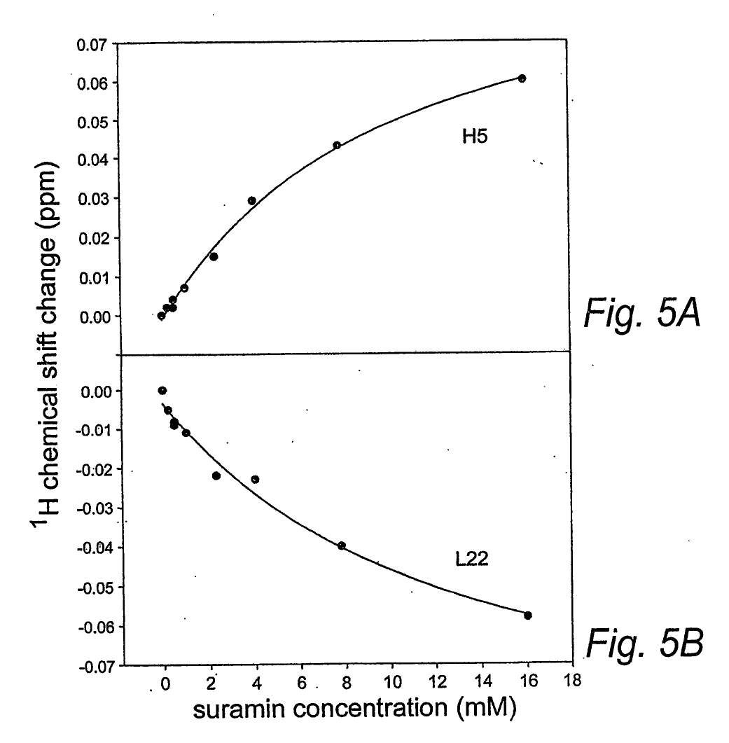

FIGS. 5A and B are plots of ¹H chemical shift change (in ppm) against suramin concentration (in mM) for residues H5 and L22 respectively of MSP1₁₉;

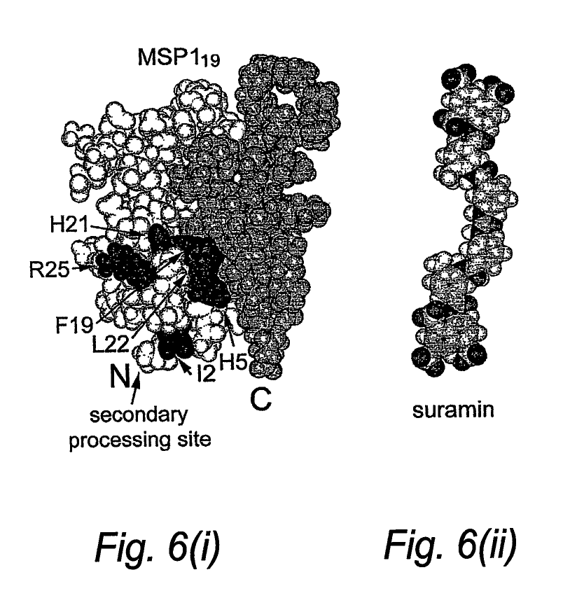

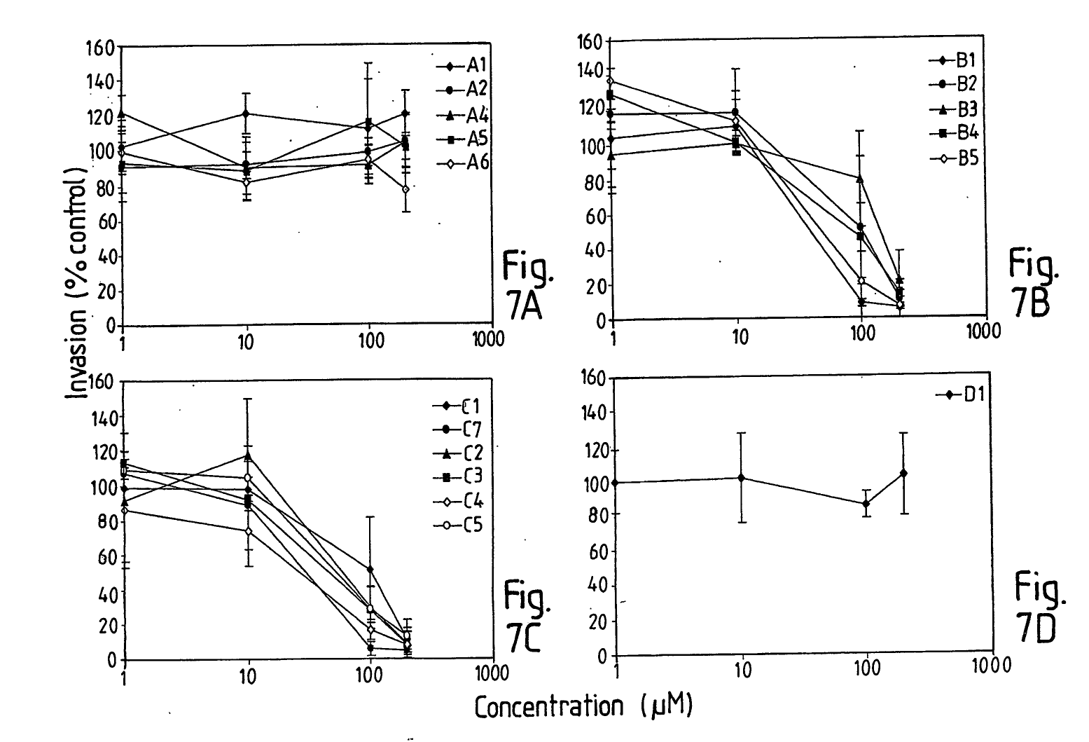

FIG. 6(i) is a representation of the 3D structure of MSP1₁₉ and FIG. 6(ii) is a representation of the 3D structure of suramin;

FIGS. 7A-D are graphs of erythrocyte invasion by merozoites (as a percentage of control samples) in the presence of different concentrations of various suramin analogues;

FIG. 8 is a sequence alignment of the amino acid residue sequence of MSP1₄₂ proteins from various Plasmodium species; and

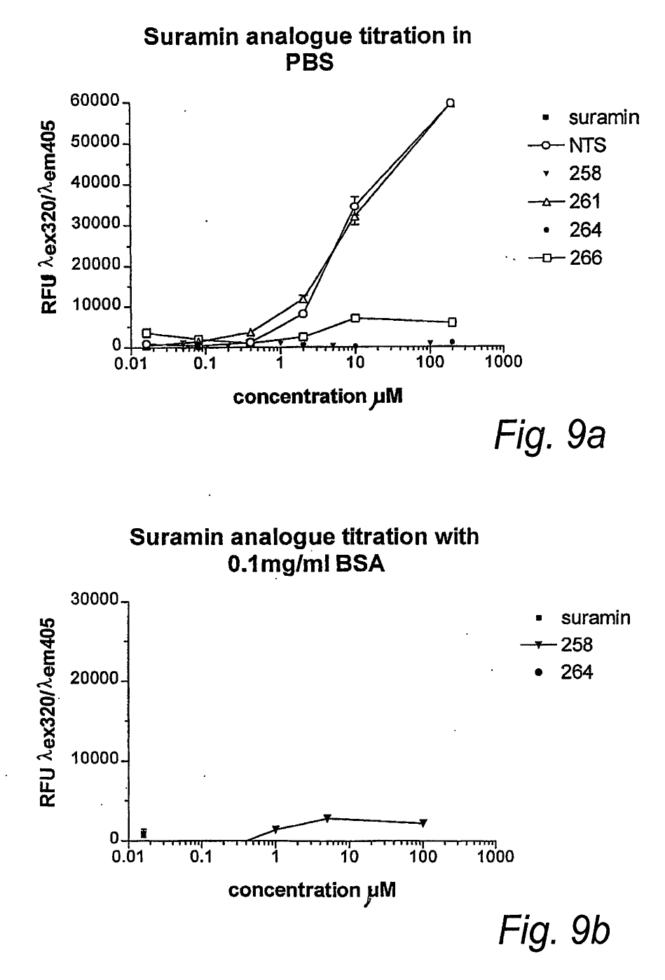

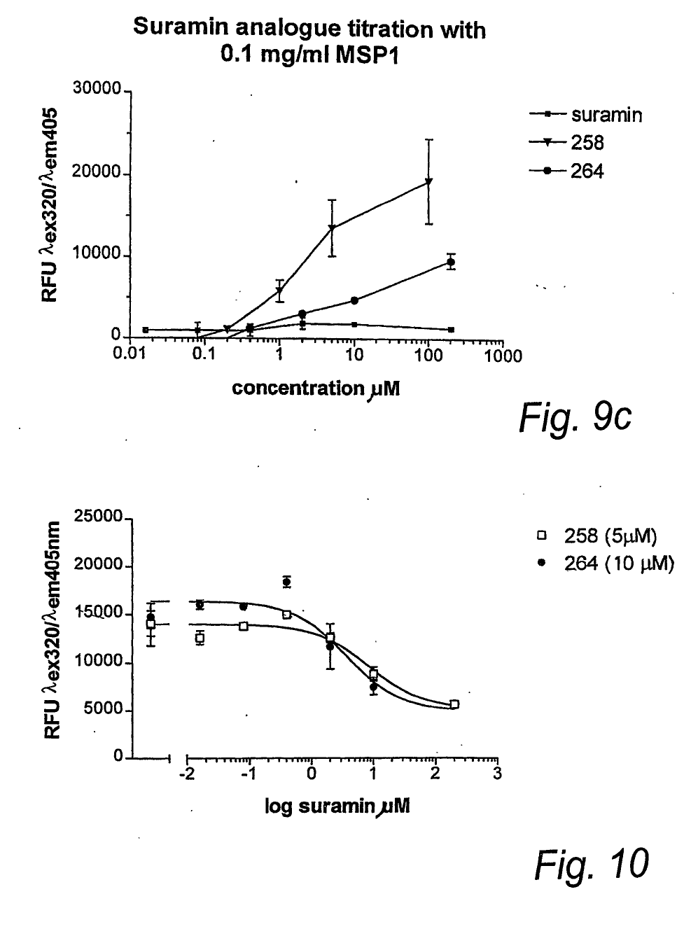

FIGS. 9a-c and 10 are graphs of fluorescence (arbitary units) against concentration of suramin or suramin analogue.

A very large number of suramin analogues are known and, for many of these, toxicity data are already available, so it would be relatively straightforward for those skilled in the art, with the benefit of the present disclosure, to identify low-toxicity analogues with suitable inhibitory properties for MSP1₄₂ processing and/or merozoite invasion of erythrocytes.

### EXAMPLES

The inventors carried out experiments to investigate what effect, if any, suramin or a suramin analogue might have on invasion of erythrocytes by merozoites.

### Example 1

Methods

Suramin, sodium salt, (Antrypol (ICI)) and suramin analogues (synthesised and described by Balaban & King in 1927, [J. Chem. Soc. 3068-3097]) (see Table 1) were provided by Mr Terry Scott-Finnigan (Division of Parasitology, National Institute for Medical Research, London, UK) and Dr Roy Bicknell (Institute of Molecular Medicine, Oxford). Data for the compounds' maximum tolerated dose (MTD) in mice is detailed by Braddock et al, (1994 Br. J. Cancer 69, 890-898). Naphthalene-1,3,6-trisulphonic acid (NTS) tri-sodium hydrated salt was purchased from Fluka (#70310).

In vitro Culture and Synchronisation of P. falciparum

Asexual blood stages of P. falciparum (FCB-1) were maintained at 37° C. in RPMI 1640/Albumax medium (Gibco) supplemented with 2 mM L-glutamine as previously described (Blackman, 1994 Methods Cell Biol. 45, 213-220). Cultures were gassed with 7% CO₂, 5% O₂ and 88% N₂ and maintained by routine passage in fresh human erythrocytes. Parasites were synchronised by Percoll and sorbitol treatment (Holder & Freeman, 1982 J. Exp. Med. 156, 1528-1538); schizonts were purified by centrifugation over Percoll and then returned to culture in the presence of fresh erythrocytes. After 4 h, during which time released merozoites invaded erythrocytes, the cells were treated with 5% sorbitol for 10 min to lyse the residual schizonts, before returning the parasites to culture.

P. falciparum In vitro Invasion and Growth Inhibition Assays

Compounds were tested for their ability to inhibit invasion in vitro, using two approaches: a short-term assay in which the number of newly invaded erythrocytes was counted using microscopy, and a growth assay measuring uptake of [³H] hypoxanthine. In the short term-assay (described previously by Blackman et al, 1990 J. Exp. Med. 172, 379-382), compounds were incubated with purified P. falciparum schizonts at ^{˜}2% parasitaemia and ^{˜}2% haematocrit in triplicate cultures. After between 6 and 24 h, blood smears were stained with Giemsa's reagent and examined by microscopy. The number of newly invaded ring stages was counted and inhibition of invasion was expressed as percent invasion relative to an untreated culture [(I_{C}/(I_{C}+U_{C})/(I_{A}/(I_{A}+U_{A})×100%], where I_{C} is the number of erythrocytes infected with ring stages, U_{C} is the number of uninfected erythrocytes in the presence of the compound, I_{A} is the number of erythrocytes infected with ring stages, and U_{A} is the number of uninfected erythrocytes in the absence of the compound.

Suramin and NTS were also assayed for P. falciparum growth inhibition using [³H] hypoxanthine uptake (generally as described by Desjardins et al, 1979 Antimicrob. Agents Chemother. 16, 710-718). Serial dilutions of the compounds in 96-well plates were incubated at 37° C. with purified mature schizonts at a final parasitaemia of ^{˜}0.5% and a haematrocrit of ^{˜}2% in triplicate cultures. Following incubation for about 24 h, [³H] hypoxanthine (0.5 Ci/well) was added for a further 18 h, and the cells were harvested onto glass fibre filters (Filtermat A, Wallac, Turku, Finland) using a cell harvester. The filters were wetted with scintillation cocktail and the bound radioactivity counted in a β-counter. Control incubations without compound or without parasitised erythrocytes were included in each experiment. The amount of radioactivity in each sample was expressed as a percentage of activity in the control wells containing no compound. Three independent experiments were performed for each compound.

### Example 1

Results

Microscopic examination of parasite cultures following staining with Giemsa's reagent suggested that suramin inhibited merozoite invasion of erythrocytes. Free merozoites were observed in the stained samples, implying that suramin did not inhibit merozoite release from schizonts. These finds were confirmed by the more quantitative [³H] hypoxanthine incorporation assay. The results (in triplicate) of the hypoxanthine assay are shown in FIG. 2. The open symbols show the results for suramin, the closed symbols are the results for NTS. The amount of radioactivity incorporated is proportional to the number of growing parasites and the effect of the compound is expressed relative to incorporation in the absence of compound (% relative parasite growth).

FIG. 2 shows that suramin inhibited erythrocyte invasion in a dose dependent manner with an IC₅₀ of 60 M+/−9, whereas NTS did not inhibit invasion even at 200 μM (the highest concentration tested).

### Example 2

In order to investigate the basis for the inhibition of invasion noted above, the inventors explored the effect of suramin and NTS on secondary processing of MSP1.

P. falciparum MSP1 Secondary Processing Assay

Merozoites were purified as described previously (Blackman 1994 Methods Cell Biol. 45, 213-220) after release from mature schizonts into growth medium supplemented with 2 mM EGTA. Merozoites were harvested by centrifugation, followed by sequential passage through pre-wetted filters of 3 and 1.2 μm pore size. Merozoites were washed in Ca²⁺- and Mg²⁺-free PBS in the presence of protease inhibitors (leupeptin, antipain and aprotinin at 10 μg ml⁻¹). The merozoites were then washed and resuspended in 50 mM Tris-HCl pH 7.2, 5 mM CaCl₂, 1 mM MgCl₂, plus leupeptin, antipain and aprotinin at 10 μg ml⁻¹ (processing buffer), and divided into 18 μl aliquots. Two microlitres of either diluted compound or reaction buffer were added and the samples incubated for 1 h at 37° C. Control assays included those in which processing was prevented by immediate addition of either SDS, 1 mM PMSF or 5 mM EGTA; and those in the absence of compound or in the presence of 0.2 mM TLCK (tosyl-_{L}-lysyl chloromethyl ketone). After 1 h the reaction was stopped, and MSP1 processing was analysed using a Western blot-based assay (Blackman 1994 Methods Cell Biol. 45, 213-220). The blots were probed with a rabbit anti-MSP1₃₃/MSP1₄₂ antibody and the bands corresponding to MSP1₄₂ and MSP1₃₃ polypeptides were visualised by enhanced chemiluminescence.

Purified merozoites were incubated in the presence or absence of suramin or NTS and polypeptides subjected to SDS-PAGE and Western blotting. The results are shown in FIGS. 3A and 3B.

FIG. 3A is a picture of blot of P. falciparum comprising: lane 1—SDS; lane 2—no further addition; lane 3—1 mM PMSF (potent protease inhibitor); lane 4—200 μM NTS; or lane 5—200 μM suramin. The major band is that corresponding to MSP1₄₂; the minor, lower band (where present) is MSP1₃₃, one of the products of secondary processing of MSP1₄₂. The absence of MSP1₃₃ (as in lanes 1, 3 and 5) is indicative of inhibition of MSP1₄₂ secondary processing.

Further experiments (results shown in FIG. 3B) demonstrated that the observed inhibition of MSP1₄₂ processing by suramin was dose-dependent. FIG. 3B shows the results of a western blot, using the assay protocol described above, with P. falciparum merozoites incubated in: lane 1—SDS; lane 2—no further addition; lane 3—1 mM PMSF; lane 4—200 μM suramin; lane 6—50 μM suramin; lane 7—12.5 μM suramin; lane 8—3.1 μM suramin and lane 9—1.25 μM suramin.

### Example 3

Fluorimetry Assay and Isothermal Titration Calorimetry

The nature of the interaction between suramin and MSP1₄₂ was investigated further. A number of MSP1-based recombinant proteins were prepared from P. falciparum and P. vivax as described below.

P. falciparum MSP-1₄₂

The pETATPf MSP1₄₂ plasmid (described by Angov et al, 2003 Mol. Biochem. Parasitol. 128, 195-204) was used to express a His₆-tagged MSP1₄₂ protein (P. falciparum 3D7 clone, Accession Number Z35327). The DNA was used to transform E. coli BL21 (DE3) cells and then clones were selected on plates of agar containing ampicillin. Cells expressing the modified MSP1₄₂ were grown to mid log phase (OD₆₀₀0.5-0.8) at 37° C., then after the incubation temperature had been reduced to 25° C., the protein expression was induced by addition of 0.1 mM IPTG. After 2 h of further growth the bacterial cells were harvested by centrifugation. The resulting cell paste was resuspended in lysis buffer (10 mM sodium phosphate, 50 mM NaCl, 10 mM imidazole, pH 6.2) and lysed by microfluidization. The final lysate was then adjusted to 500 mM NaCl and 1% (v/v) Tween 80 (final concentrations) and incubated for a further 20 min with mixing. After centrifugation at 30,000×g for 1 h the supernatant was applied to a column containing Ni²⁺ NTA Superflow resin (Qiagen). The column was washed extensively and sequentially with the following buffers: 10 mM sodium phosphate, 500 mM NaCl, 10 mM imidazole, 0.5% Tween 80, pH 6.2; 10 mM sodium phosphate, 75 mM NaCl, 20 mM imidazole, pH 8.0. Bound protein was eluted with 10 mM sodium phosphate, 75 mM NaCl, 160 mM imidazole, pH 8.0.

P. falciparum MSP₁₁₉

Plasmodium falciparum MSP1₁₉ (Swiss-Prot 04933) was expressed from a synthetic gene optimised for Pichia pastoris expression (European Patent No. EP1180120). This construct, inserted at the SnaBI site of vector pPIC9K (Invitrogen), contained the N-terminal leader sequence YHHHHHHIEGRH preceding the MSP1₁₉ sequence. A point mutation (Ser3 to Ala) was also introduced to eliminate N-glycosylation at Asn1. Following purification of MSP1₁₉ as described previously (Morgan et al, 1999 J. Mol. Biol. 289, 113-122), the N-terminal tag was cleaved with Factor Xa (New England Biolabs) and MSP1₁₉ was purified by gel filtration (Superdex 200). The fmal product contained the 96 amino acid MSP1₁₉ fragment, preceded by a single His residue.

The MSP1₄₂ protein from P. vivax was cloned into plasmid jmp28, a modified pET-28 vector that encodes an N-terminal peptide MHHHHHHIEGRWIL immediately upstream of the inserted sequence. P. vivax (Belem strain) DNA was used as a template for PCR based cloning. The sequence of the expressed protein (following the vector encoded N-terminal peptide) corresponded to residues Asp 1325 to Ser 1704, Accession number A39401.

The protein was expressed in BL21(DE3)pLysS cells by induction of a culture at an OD₆₀₀ of 0.6-0.8 with IPTG at a final concentration of 1 mM. The cells were induced for 3 h at 37° C. and then harvested by centrifugation. The cells were lysed using the Bugbuster protein extraction reagent (Novagen), after centrifugation of the cell lysate, the pellet containing the MSP1₄₂ was dissolved in 6M Guanidine-HCl, 100 mM Na₂HPO₄, 10 mM Tris-HCl, pH 8.0. This was applied to a Ni-NTA column, washed with 10 column volumes of the loading buffer, followed by 6 column volumes of 8 M Urea, 100 mM Na₂HPO₄, 10 mM Tris-HCl, at pH 7.0, and 6 column volumes at pH 6.1. The protein was eluted in 3 column volumes 8 M Urea, 100 mM Na₂HPO₄, 10 mM Tris-HCl, pH 4.3.

The protein was refolded by rapid dilution (1:50) into refolding buffer (55 mM Tris-HCl, pH8.2, 264 mM NaCl, 11 mM KCl, 550 mM guanidinium hydrochloride, 1.1 mM EDTA, 1 mM GSH [glutathione], 0.1 mM GSSG [glutathione, oxidised form]) and incubation overnight at 18° C. The protein was concentrated, then purified by gel filtration on a Superdex 200 (26/600 mm) column (Amersham) equilibrated in 20 mM Tris-HCl, 250 mM NaCl, pH 8.0. Protein containing peaks were pooled, diluted 5-fold into 20 mM Tris-HCl, pH 8.0 and applied to a Mono-Q HR 5/5 column (Amersham) and eluted using a NaCl gradient from 0-250 mM. The protein eluted at 150-250 mM NaCl. The fractions containing MSP1₄₂ were pooled and dialysed extensively with PBS.

P. vivax MSP1₃₃

P. vivax MSP1₃₃ was expressed using the P. vivax MSP1₄₂ clone, above, as a template for PCR, followed by ligation of the product into vector pET30Xa/LIC. The sequence runs from Asp 1325 to Ser 1618 (Accession number A39401) following the N-terminal purification tag. The vector was used to transform BL21(DE3)pLysS cells. For expression, a culture of the transformed cells was induced at an OD₆₀₀ of 0.6-0.8 at 37° C. with 1 mM IPTG for 3 h. The cells were harvested by centrifugation and the cell pellet lysed using the Bugbuster protein extraction reagent (Novagen). The cell lysate was cleared by centrifugation and the supernatant loaded directly onto a Ni-NTA column. The column was washed with 10 volumes of 50 mM NaH₂PO₄, 300 mM NaCl, 10 mM imidazole, pH 8.0, then with 6 column volumes of the same buffer containing 30 mM imidazole, and finally eluted with 3 column volumes of the buffer with 250 mM imidazole. The eluted protein was then loaded directly onto a Superdex 200 (26/600) column (Amersham) equilibrated in 20 mM Tris-HCl, 250 mM NaCl, pH 8.0 and purified by gel filtration. The fractions containing MSP-1₃₃ were pooled and dialysed extensively with PBS.

Binding of Suramin to MSP1 Assayed by Fluorimetry

The intrinsic fluorescence of suramin was used to determine its capacity to bind to the various MSP1-derived proteins. The fluorescence was measured using a Perkin Elmer LS-3B or a Spex Fluoro Max 2 fluorimeter. Suramin was excited at a wavelength of 315 or 330 nm with 2.5 nm resolution, the emission spectra were measured at wavelengths ranging from 350-450 nm. Suramin and the MSP1 proteins were diluted in 20 mM NaH₂PO₄, 150 mM NaCl at pH 7.2 and analysed at 10 or 20° C. Titrations were performed by adding aliquots of the suramin solution to the MSP1 solution. In controls, suramin was titrated into buffer. The K_{d} for the suramin-MSP1 binding was determined from three independent experiments.

Typical results are shown in FIG. 4. The initial solution contained 0.3 μM MSP1₄₂, 20 mM NaH₂PO₄, 0.15M NaCl pH 7.2 at 20° C. to which μ1 aliquots of 100 μM suramin were added.

The intrinsic fluorescence of suramin is low when excited at 315 nm, but when it binds to MSP1₄₂ it shows a pronounced increase in emission intensity with the maximum emission slightly shifting from 408 to 411 nm (FIG. 4 insert). This fluorescence enhancement was used to measure binding of suramin to MSP1₄₂ from P. falciparum. Following titration of a solution of MSP1₄₂ with suramin, the marked increase in the fluorescence intensity was measured as a function of suramin concentration (FIG. 4). Analysis of the binding data revealed that suramin binds to MSP1₄₂ from P. falciparum with a K_{d} of 0.22 μM ±0.04.

In addition to the fluorimetry, isothermal titration calorimetry (ITC) was performed using a Microcal omega VP-ITC (MicroCal Inc., Northampton, Mass.). The proteins were dialyzed extensively against the ITC buffer (phosphate buffered saline, pH 7.4). All experiments were performed at 25° C. The cell, 1.425 ml, contained 30 μM MSP1₄₂ or MSP1₃₃ and these were titrated by injection of a total of 290 μl of 600 μM suramin. The heat of dilution of suramin into buffer was determined in control experiments. The data were fitted by least-squares methods using the evaluation software, Microcal Origin version 5.0 provided by the manufacturer. Each experiment was performed twice.

MSP1₄₂ and MSP1₃₃ from P. vivax were prepared as described above and their suramin binding properties analysed by isothermal titration calorimetry. MSP1₃₃ from P. falciparum was soluble to only 0.15 mg/mL in aqueous buffers and aggregated significantly even at these concentrations, preventing measurements of suramin binding in vitro. However MSP1₃₃ from P. vivax was soluble to at least 20 mg/mL and hence amenable to in vitro methods of measuring suramin binding. P. vivax MSP1₄₂ was found to bind suramin with a similar K_{d} (0.3±0.1 μM) to that measured for P. falciparum MSP1₄₂. MSP1₃₃ from P. vivax showed a high affinity site that was 5 fold weaker than for MSP1₄₂ (1.5±0.5 μM). The AH values (11±1 kcal/mol for MSP1₄₂ and 12±1 kcal/mol for MSP1₃₃) are also very similar. At the high concentrations (>30 μM) required for ITC analysis there was evidence for some non-specific binding. When analysed by fluorimetry P. vivax MSP1₄₂ and MSP1₃₃ showed large binding-induced enhancements of the intrinsic suramin fluorescence similar to that observed with P. falciparum MSP1₄₂. Thus P. falciparum MSP1₄₂, P. vivax MSP1₄₂ and P. vivax MSP1₃₃ all exhibited large enhancements of suramin fluorescence on binding. The suramin binding for MSP1₃₃ is only fivefold weaker than that for MSP1₄₂ and this taken together with the similar large induced fluorescence enhancements indicates that there is a similar hydrophobic suramin binding pocket in the two proteins.

### Example 4

NMR Studies with MSP1₁₉

No fluorescence enhancement was observed when suramin was added to MSP1₁₉ from P. falciparum and thus suramin binding to MSP1₁₉ could not be determined by fluorescence measurements. However, using NMR it was possible to follow the changes in chemical shifts of the ¹H and ¹⁵N signals in ¹H-¹⁵N HSQC experiments when titrating ¹⁵N labelled MSP1₁₉ with suramin.

NMR experiments were carried out on Varian spectrometers operating at proton frequencies of 500, 600 and 800 MHz. Suramin (0.03-16.0 mM) ¹H spectra were recorded at 5 to 35° C. The assignments of the ¹H signals of suramin (except for the NH signals) were made by analysis of the 2D gradient selected double quantum filtered COSY spectrum at 500 MHz, 25° C. on a sample containing 1 mM suramin in 50 mM potassium phosphate, 100 KCl, 90%/H₂O/10% D₂O at pH 6.5). The NH assignments were made on the basis of NOESY (nuclear Overhauser effect spectroscopy) experiments. MSP1₁₉/suramin samples were examined with either unlablled or ¹⁵N labelled MSP1₁₉ in 50 mM sodium phosphate and 100 mM NaCl in 90% H₂O/10% D₂O at pH 6.5 (pH values are pH meter readings uncorrected for deuterium isotope effects). A titration was carried out by mixing two samples each containing 0.1 mM MSP1₁₉ and with one also containing 16 mM suramin. 1D ¹H and 2D ¹H-¹⁵N HSQC NMR spectra were recorded for each concentration of suramin. NOESY spectra were recorded on MSP1₁₉/suramin samples in D₂O (1.76 mM protein and 6 mM suramin in 50 mM sodium phosphate and 100 mM NaCl at pH 6.5); these spectra were compared to MSP1₁₉ spectra recorded in the absence of suramin to detect any suramin induced changes in ¹H chemical shifts for protein side chain resonances.

At the maximum concentration of suramin used, 16 mM, about 50% of MSP1₁₉ was complexed with the ligand. The residues that showed the largest shifts on addition of suramin were Ile 2, His 5, Phe 19, His 21, Leu 22 and Arg 25. The shifts were fitted by non-linear regression analysis to a single binding curve and gave an average K_{d} ^{˜}15 mM±5.

FIG. 5A shows binding curves illustrating the suramin concentration dependence of the ¹H chemical shifts of the NH signals from His 5 and Leu 22 residues of MSP1₁₉.

FIG. 6(i) is a representation of the structure of MSP1₁₉ as determined by nmr (Morgan et al, 1999 J. Mol. Biol. 289, 113-122). The residues affected by suramin binding are shown in black. N and C indicate the sites of the protein termini.

FIG. 6(ii) shows suramin in its extended conformation, to the same scale, for comparison.

TABLE 1

| | Urea derivative (R) | | |
|---|---|---|---|
| Naphthylamine derivative | 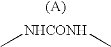 | 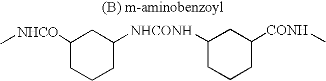 | 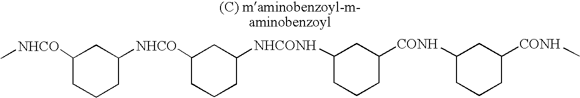 |
| 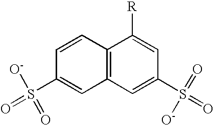 | A1 (CPD 6) | B1 | C1 |
| 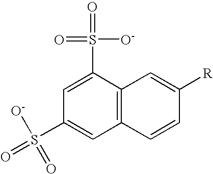 | A2 (CPD 1) | B2 (CPD 9) | C2 (CPD 10) |
| 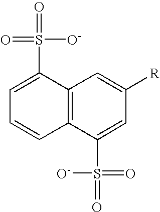 | A3 (CPD 3)* | B3 | C3 (CPD 11) |
| 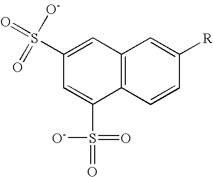 | A4 (CPD 4) | B4 (CPD 12) | C4 (CPD 13) |
| 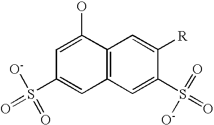 | A5 (CPD 7) | B5 (CPD 14) | C5 (CPD 15) |
| 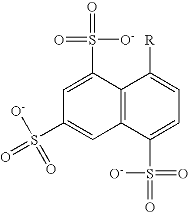 | A6 (CPD 2) | | C6 (CPD 16)* |
| 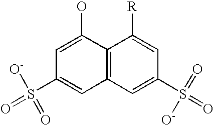 | | | C7 (CPD 8) |

| | | |
|---|---|---|
| 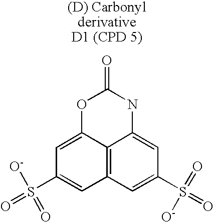 | 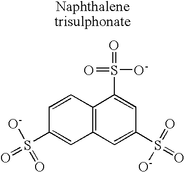 | 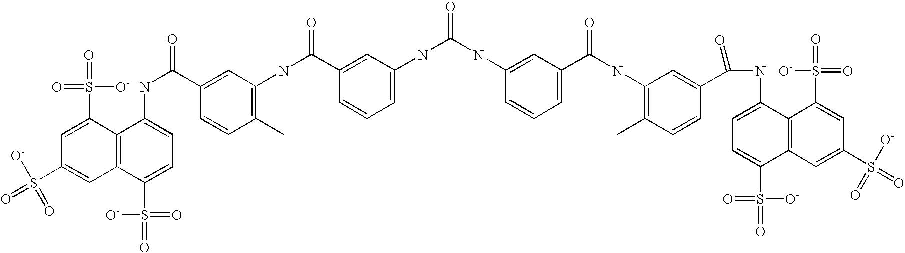 |

*not tested

### Example 5

Suramin Analogues

Suramin Analogues Inhibit Erythrocyte Invasion and MSP1 Secondary Processing

A number of suramin analogues were examined in order to probe the features of the molecule necessary for binding to MSP1 and for inhibiting P. falciparum MSP1 processing. Four series of symmetrical compounds (Groups A-D, Table 1) differing in the number of central aminobenzoyl urea units, and having various substitutions on the terminal naphthyl rings, were examined.

The results of invasion assays are shown in FIGS. 7A-D, which are graphs of erythrocyte invasion (as a percentage of control experiments in the absence of analogue) against concentration of analogue (μM). The error bars denote the standard deviation.

None of the Group A compounds or the single group D compound (D1) inhibited invasion in vitro at 200 μM, the highest concentration tested (FIG. 7A and D). However, Group B and C compounds inhibited invasion in vitro (FIG. 7B and C), with IC₅₀ values similar to that of suramin. The suramin analogues differ in toxicity. The least toxic inhibitory compound, B1, is 10 times less toxic than suramin. The various suramin analogues were also examined for their ability to inhibit MSP1₄₂ processing, as described above. These assay results (omitted for brevity) demonstrated that the compounds that inhibit invasion also inhibit MSP1₄₂ processing, whereas those that do not inhibit invasion also do not inhibit processing, when tested at the concentration of 200 μM. Similar parasite invasion and MSP1 processing assays could not be carried out for P. vivax since this species cannot be cultured in vitro.

### Example 6

Fluorescence Properties of Suramin Analogues

A number of suramin analogues known to inhibit MSP1 processing at 200 μM were investigated. Naphthalenetrisulphate was included as a negative control. The analogues investigated are shown below.

| Number | Original code* | Side group | linker |
|---|---|---|---|
| 254 | C7 (CPD8) | H-acid | m'aminobenzoyl-m-aminobenzoyl |
| 258 | C2 (CPD10) | Amino-G | m'aminobenzoyl-m-aminobenzoyl |
| 261 | C3 (CPD10) | C-acid | m'aminobenzoyl-m-aminobenzoyl |
| 264 | C4 (CPD13) | Amino-J | m'aminobenzoyl-m-aminobenzoyl |
| 266 | B5 (CPD14) | 2nR-acid | m'aminobenzoyl |

*Used in Table 1

### Example 6A

Inhibition of Suramin Binding

Method

MSP1₄₂ (0.1 mg/ml), 10 μM suramin analogue and 2 μM suramin were combined in a 384-well black polypropylene low binding plate (total reaction volume 100 μL). Fluorescence was read at λₑₓ 320 nm/λₑₘ 405 nm on a BMG Polarstar fluorimeter. Readings were taken before, and 1 hour after, addition of suramin.

Results

All compounds tested interfered with the fluorescent signal of suramin at 10 μM, but no inhibition of suramin binding could be detected. Analogue 254 served to quench suramin mediated fluorescence. Analogues 258, 261, 264 and 266 were all fluorescent. Naphthalenetrisulphate was the most fluorescent compound tested.

Conclusions

The low suramin fluorescence signal (signal to noise ratio 1.8) is suceptible to interference test compounds and is not robust enough for high through-put screening. The high rescent signals observed for analogues 258, 216, 264 and 266 (signal to noise ratios in range 4-20) may provide an alternative approach.

### Example 6B

Titration of Fluorescent Analogues—MSP1 Specificity

Method

Analogues 258, 261, 264 and 266 were titrated against (a) PBS buffer only, (b) 0.1 mg/ml bovine serum albumin in PBS or (c) 0.1 mg/ml MSP1₄₂ in PBS. Fluorescence observed was compared to naphthalenetrisulphate (non-specific reagent) and suramin. Analogue concentrations in the range 0.01-200 μM were tested and the results are shown in FIGS. 9(a)-(c) respectively.

FIGS. 9a-c are graphs of relative fluorescence units (“RFU”) against concentration (μM) of suramin and various suramin analogues in the presence of PBS (FIG. 9a), 0.1 mg/ml BSA in PBS (FIG. 9B), or 0.1 mg/ml MSP1₄₂ (FIG. 9c).

In the graphs, plots for suramin are denoted by black squares, naphthalenetrisulphate by white circles, analogue 258 by black triangles, analogue 261 by white triangles, analogue 264 by black circles, and analogue 266 by white squares.

Results

Analogues 261 and 266, like naphthalenetrisulphate, were fluorescent in buffer alone, and so were disregarded from further testing. Analogues 258 and 264, like suramin, became fluorescent in the presence of MSP1₄₂ specifically. Analogue 258 shows low levels of fluorescence in the presence of BSA (a non-specific binding protein) at high concentrations, but this is 10-fold less than that observed with MSP1. This level of non-specific fluorescence is not high enough to significantly affect an assay result and can be disrgarded.

Analogues 258 and 264 have lower affinity for MSP1₄₂ than suramin (EC50 ^{˜}10 μM or greater, compared to EC50 ^{˜}2 μM), but show much greater fluorescent change upon binding to MSP1 (10-20 fold higher than suramin). This fluorescent change could be exploited to screen for inhibitors of MSP1 processing.

### Example 6C

Competition Binding Analogues 258 and 264 Against Suramin

Method

To demonstrate the use of analogues 258 or 264 as a screen for inhibitors of the MSP1 suramin binding site a competition binding experiment with suramin was set up. MSP1₄₂ (0.1 mg/ml) and fixed concentrations of analogue 258 (5 μM) or 264 (10 μM) were premixed and 90 μL added to a 384-well black polypropylene plate. Suramin was added to give a final concentration in the range 0.01 to 200 μM in a total reaction volume of 100 μL. The plates were incubated at room temperature protected from light for 1 hour, and then read as before. Results are shown in FIG. 10, which is a graph of fluorescence (arbitary units) against concentration of suramin competitor for analogue 258 (white squares) and 264 (black circles).

Results

Suramin was able to displace both analogue 258 and analogue 264 from MSP1, and a concomitant decrease in fluorescence was observed. Fitting the data to a sigmoidal dose response curve (using Graphpad prism) an IC50 of 8 and 3 μM was estimated for 258 and 264 respectively.

### Example 7

This example relates to an illustration of an assay suitable for performing the screening method of the invention.

In the example, the comparison substance is suramin, but many other compounds (especially analogues of suramin of groups B and C in Table 1) could be employed and may indeed be preferred. Suramin is convenient as it possesses intrinsic fluorescence which, upon binding to MSP1₄₂ or a fragment thereof, is greatly enhanced and this enhanced fluorescence can be used as a measure of MSP1₄₂-binding.

The assay is performed in a conventional microtitre plate. The wells of the plate comprise purified MSP1₄₂, derived from P. falciparum or P. vivax at an appropriate concentration (e.g. about 1 μM). To respective individual wells (perhaps in duplicate or triplicate) the test substances would be added, typically at a concentration of about 5-500 μM preferably about 10-50 μM. At concentrations above about 500 μM a large number of compounds might exhibit non-specific binding, which is not of interest from a clinical viewpoint. If desired the test substances may be tested at a number of different concentrations. A fluorescence measurement is then performed, with excitation at about 330 nm and detecting emission at about 440 nm. (With suramin the precise wavelengths used are not very important, as the excitation/emission spectra are very broad: thus, excitation could be performed at any wavelength in the range 305-375 nm and emission could be detected over the range 380-450 nm).

This initial fluorescence measurement will detect those test substances which are themselves naturally fluorescent at a relevant wavelength (or become so upon binding to MSP1₄₂ or a fragment thereof), which can then be allowed for when interpreting the assay results.

Suramin, the comparison substance, is then added to the wells at a suitable concentration, which will typically be less than the concentration of the test substance. Typically the concentration of the composition substance will be in the range 0.5 μM-10 μM. The microtitre plate is then left to incubate a suitable period of time at a suitable temperature (e.g. 1 hr at 20° C., or say 30 minutes at 37° C.) to allow any reaction to proceed. A second fluorescence measurement is then made (typically using the same excitation/emission wavelengths as for the first measurement). Those wells in which the suramin or suramin analogue mediated fluorescence is abolished or significantly reduced indicates that the test substance has bound to MSP1₄₂ (or the fragment thereof) at the same site as would have been occupied by suramin and has therefore prevented suramin binding.

Test substances of potential interest identified in this way may then be made the subject of further analysis and investigation, for example by assay in the MSP1 secondary processing and/or inhibition of invasion tests as described herein.

1. A method of screening a test substance for possession of binding activity for MSP1₄₂ or a fragment thereof, the method comprising the steps of: combining or contacting, in any order,
(i) a molecule comprising MSP1₄₂ or a fragment thereof,
(ii) the test substance, and
(iii) a comparison substance known to have binding activity for MSP1₄₂ or a fragment thereof;
and determining the presence and/or amount, if any, of comparison substance and/or test substance bound to the MSP1₄₂ or fragment thereof.

2. A method according to claim 1, wherein the comparison substance inhibits processing of MSP1₄₂ and/or inhibits merozoite invasion of erythrocytes.

3. A method according to claim 1, wherein the comparison substance is suramin or a suramin analogue.

4. A method according to claim 1 wherein the comparison substance and/or the test substance is labelled to facilitate detection.

5. A method according to claim 1 wherein the fragment of MSP1₄₂ comprises MSP1₁₉ and MSP1₃₃.

6. A method according to claim 1 wherein binding of the comparison and/or test substance is determined by fluorescence measurements.

7. A method according to claim 1, wherein the test substance is screened against a number of different MSP1₄₂ molecules or fragments thereof.

8. A method according to claim 1, wherein the MSP1₄₂ moelcule or fragment thereof is a mutant of a naturally-occurring wild type sequence.

9. A method according to claim 1, wherein the comparison substance comprises a suramin analogue having a m-aminobenzoyl or m′-aminobenzoyl-m-aminobenzoyl moiety.

10. A method according to claim 1, wherein the comparison substance comprises a suramin analogue which is symmetrical.

11. A method according to claim 10, wherein the analogue substance comprises suramin analogues C2 and C4 as defined in Table 1 herein, and having the structure shown below:

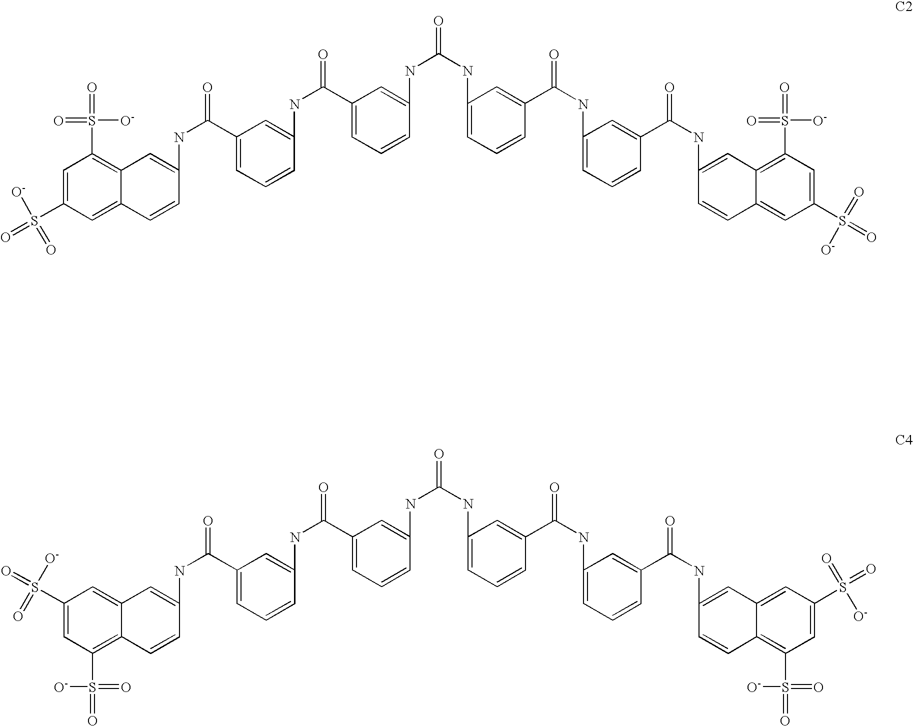

12. A method according to claim 1, wherein the comparison substance comprises a molecule which exhibits at least a twofold increase in fluorescence upon binding in MSP1₄₂ or a fragment thereof.

13. (canceled)

14. A pharmaceutical composition comprising suramin or an analogue thereof and a pharmaceutically effective carrier therefor for use in the prevention and/or treatment of malarial disease in a mammalian subject.

15. A pharmaceutical composition, comprising suramin or an analogue thereof and a pharmaceutically effective carrier therefor for use in the prevention and/or treatment of malarial disease in a mammalian subject wherein the active ingredient is identified by performance of a method in accordance with claim 1.

16. (canceled)

17. A method of treating or preventing malarial disease in a mammal subject to said disease which comprises administering to said mammal an effective amount of a composition according to claim 14.
